(19) United States
(12) Patent Application Publication
(10) Pub. No.: US 2010/0222309 A1
(43) Pub. Date: Sep. 2, 2010

(54) SKIN PROPERTY-IMPROVING THERAPEUTIC AGENT COMPRISING A MORPHINAN DERIVATIVE OR ANY OF ITS PHARMACOLOGICALLY PERMISSIBLE ACID ADDITION SALTS AS AN ACTIVE INGREDIENT

(75) Inventors: Shinji Ona, Tokyo (JP); Koichiro Takeshita, Tokyo (JP); Naoki Ando, Tokyo (JP); Masahiro Yamashita, Tokyo (JP)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: TORAY INDUSTRIES, INC., Tokyo (JP)

(21) Appl. No.: 12/680,908

(22) PCT Filed: Oct. 3, 2008
(86) PCT No.: PCT/JP2008/068092

§ 371 Date: Mar. 31, 2010

(30) Foreign Application Priority Data
Oct. 5, 2007 (JP) 2007-262015

(51) Int. Cl. A61K 31/56; A61K 31/485; A61P 17/00

(52) U.S. Cl. 514/171; 514/282

(57) ABSTRACT

A skin property-improving therapeutic agent includes a compound having a specific morphinan skeleton typified by a compound 1 represented by the following formula (II) or any of its pharmacologically permissible acid addition salts as an active ingredient:

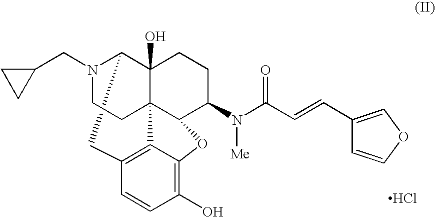

### RELATED APPLICATIONS

This is a §371 of International Application No. PCT/JP2008/068092, with an international filing date of Oct. 3, 2008 (WO 2009/044883 A1, published Apr. 9, 2009), which is based on Japanese Patent Application No. 2007-262015, filed Oct. 5, 2007, the subject matter of which is incorporated by reference.

### TECHNICAL FIELD

This disclosure relates to the provision of a novel medicine comprising a morphinan derivative or any of its pharmacologically permissible acid addition salts useful for therapy to improve skin properties via a skin moisture retaining effect.

### BACKGROUND

It is known that skin roughening, skin drying, skin darkening and the like are caused by the functional decline of the skin barrier, and for the purpose of improving skin properties, toilet water, foundations, emulsions, horse oil, ointments and the like have been being used. As skin property-improving medicines, medicines for external application including heparinoids such as Hirudoid, Airleet, Kuradoid, Seleloiz and Besoften, urea, hyaluronic acid and collagen are formulated for treating such indications as asteatosis cutis and keratodermia tylodes palmaris progressiva. Further, it is also reported that cosmetics comprising, as a main ingredient, an extract obtained by extracting the roots, subterranean stems or leaves of Phyllostachys bambusoides, Phyllostachys nigra or Phyllostachys heterocycle using a hydrophilic organic solvent (JP 5-124930 A) and cosmetics comprising, as a main ingredient, an extract of bamboo stems or branches (JP 7-187990 A) are effective for improving the dry skin accompanied by astetic change. It is also reported that some plant extracts promote the liberation of β-endorphins from keratinocytes, for promoting the moisture retention of the skin (“New Role of β-endorphin in Skin (in Japanese),” (Mika Adachi et al.), Fragrance Journal, Fragrance Journal Ltd., Jun. 15, 2005, Vol. 33, No. 6, pages 35-38).

In general, in the healthy horny layer of the skin, the stratum corneum intercellular lipids form a double lipid layer for retaining water, and since the low molecular water soluble substances in the keratin cells keep flexibility, the sebum inhibits percutaneous water transpiration, to keep the skin surface lubricating. Ceramides play important roles of retaining skin water and maintaining the barrier function of the horny layer, and in an alkaline region, it is considered that ceramidase activity rises to promote the hydrolysis reactions from ceramides into fatty acids and sphingosine, resulting in dry skin (“Purification and Biochemical Characterization of Membrane-bound Epidermal Ceramidases from Guinea Pig Skin,” (Yukihiro Yada et al.), The Journal of Biological Chemistry, USA, American Society for Biochemistry and Molecular Biology, May 26, 1995, Vol. 270, No. 21, pages 12677-12684). About 60% of the patients treated with hemodialysis are found to suffer from itching (“Itching up date: Effectiveness of κ-opioid Agonists for Itching of Dialysis Patients (in Japanese),” (Hironari Kumagaya et al.), M B Derma, Zen-Nihon Byoin Shuppan-kai (=Publishing Association of All Japanese Hospitals), Aug. 25, 2005, Vol. 104, pages 59-64 and “Uraemic Pruritus,” (Idit F. Schwartz et al.), Nephrology, Dialysis, Transplantation, official publication of the European Dialysis and Transplant Association—European Renal Association, United Kingdom, Oxford University Press, 1999, Vol. 14, No. 4, pages 834-839), and on the other hand, about 90% of them are found to experience dry skin and to record significant decline in the water content of the horny layer and significant rise in the pH value of the skin (“Cutaneous Lesions of Dialysis Patients. General Remarks (in Japanese),” (Akira Hattori), Rinsho Toseki (=Clinical Dialysis), Nihon Medical Center, October 1995, Vol. 11, No. 13, pages 1877-1881). Further, in addition to these, also in the xeroderma most frequently observed with the patients treated with hemodialysis, the amount of skin surface lipids is found to decrease (“Substances Relating to Complications Observed with Uremia and Chronic Dialysis X) Skin Disorders (in Japanese),” (Akira Hattori), Rinsho Toseki (=Clinical Dialysis), Nihon Medical Center, Aug. 10, 1995, Vol. 21, No. 9, pages 1237-1242), and it is also reported that these patients are higher than healthy persons in the average pH value of the skin and can have itching reduced if they are coated with an acid cream having a lactic acid buffer as an aqueous phase (“Skin Changes in Hemodialysis Patients (in Japanese),” (Akira Hattori et al.), Rinsho Hifuka (=Clinical Dermatology), Igaku-Shoin Ltd., January 1990, Vol. 44, No. 1, pages 21-24). Skin drying is often accompanied by aging, atopic dermatitis and the xeroderma occurring especially in winter, and there is a demand for the development of a moisture retaining agent that has both the water retaining function and the barrier function.

The morphinan compound used as an active ingredient displays opioid κ agonism and the applications of the morphinan compound based on its analgesic activity, diuretic activity and antitussive activity are already disclosed (WO 93/015081 A). Further, already disclosed are applications as brain cell protective (WO 95/003307 A), antipruritic agent (WO 98/023290 A), hyponatremia remedy (WO 99/005146 A), ORL-1 receptor antagonist (JP 2000-53572 A), remedy for neuropathic pain (WO 01/014383 A), remedy for psychoneurosis (WO 02/078744 A), remedy for drug addiction (WO 99/011289 A), remedy for sepsis (WO 02/089845 A), antipruritic agent for pruritus caused by multiple sclerosis (WO 06/095836 A) and the like. WO 98/023290 A discloses the antipruritic effect by K agonist via the central function.

With regard to other opioid-based drugs such as morphine than those described above, the drugs, the skin improving effects of which are disclosed, include the following. It is reported that opioids for external application are effective for sebaceous gland disorders such as acne and burns (U.S. Pat. No. 5,834,480) and that β-endorphine are capable of increasing the water content of the skin, improving the barrier function of the skin, promoting the turnover, and preventing the initial progression of aging (“New Role of β-endorphin in Skin (in Japanese),” (Mika Adachi et al.), Fragrance Journal, Fragrance Journal Ltd., Jun. 15, 2005, Vol. 33, No. 6, pages 35-38). It was found in 1999 that β-endorphins exist in the skin, and it is reported that in vitro, the β-endorphins (1) increase natural moisturizing factors (amino acids, filaggrin), (2) increase a turnover regulatory factor (cytokeratin 1), (3) increase a cell adhesion factor (desmosome), (4) increase the envelope of the horny layer (involucrin, loricrin), and (5) increase an anchoring complex (laminin 5). With regard to the effects of opioids on skin cells, it is reported that β-endorphins as an agonist enhance the production of cytokeratin 16 as a differentiation marker of epidermis cornification cells (“β-endorphin Stimulates Cytokeratin 16 Expression and Down-regulates μ-opiate Receptor Expression in Human Epidermis,” (Mei Bigliardi-Qi et al.), The Journal of Investigative Dermatology, USA, Nature Publishing Group, March 2000, Vol. 114, No. 3, pages 527-532) and that β-endorphins stimulate the wandering of keratinocytes (“μ-opiate Receptor and Beta-endorphin Expression in Nerve Endings and Keratinocytes in Human Skin,” (Mei Bigliardi-Qi et al.), Dermatology, Switzerland, Karger, 2004, Vol. 209, No. 3, pages 183-189).

It is known that opioids have analgesic action and on the other hand also function as a chemical mediator of itching, and it is reported that endogenous opioid peptides such as β-endorphins and enkephalins cause itching (“Experimental and Clinical Pruritus. Studies on Some Putative Peripheral Mediators. The Influence of Ultraviolet Light and Transcutaneous Nerve Stimulation,” (B. Fjellner), Acta Dermato-venereologica. Supplementum, Norway, Scandinavian University Press, 1981, Vol. 97, pages 1-34). Though details are not clear, it is generally said that μ-receptors and δ-receptors participate in inhibiting pain and inducing itching and that κ-receptors participate in inhibiting pain and itching (“Opioid Peptide Targeting Treatment of Pruritus (in Japanese),” (Kenji Takamori), Rinsho Hifuka (=Clinical Dermatology), Igaku-Shoin Ltd., Apr. 10, 2002, Vol. 56, No. 5, pages 145-147). Opioid-based drugs reported to have an antipruritic effect include naloxone and naltrexone (“Effects of Naltexone on Spontaneous Itch-associated Responses in NC Mice with Chronic Dermatitis,” (Tatsuya Maekawa et al.), Japanese Journal of Pharmacology, The Japanese Pharmacological Society, Oct. 1, 2002, Vol. 90, No. 2, pages 193-196 and “Itch-associated Response Induced by Experimental Dry Skin in Mice,” (Takayuki Miyamoto et al.), Japanese Journal of Pharmacology, The Japanese Pharmacological Society, Mar. 1, 2002, Vol. 88, No. 3, pages 285-292) and it is reported that specific morphinan compounds as active ingredients exhibit an antipruritic effect (“Imbalance in Opioid System as a Cause of Uremic Pruritus and Effect of a Novel κ-Agonist, TRK-820 (in Japanese),” (Hironari Kumagaya et al,), Sogo Rinsho (=General Clinical Medicine), Nagai Shoten Co., Ltd., May 1, 2004, Vol. 53, No. 5, pages 1678-1684, “Involvement of Central μ-opioid System in the Scratching Behavior in Mice, and the Suppression of It by the Activation of κ-opioid System,” (Hideo Umeuchi et al,), European Journal of Pharmacology, Netherlands, Elsevier Science, 2003, Vol. 447, No. 1, pages 29-35 and “Anti-Pruritic Effect of a Kappa Opioid Receptor Agonist TRK-820,” (Hideo Umeuchi et al.,), Journal of Pharmacological Sciences, The Japanese Pharmacological Society, Mar. 1, 2003, Vol. 91, Supplement No. 1, page 198). Further, among the drugs having μ-receptor blocking activity, some are reported to be used for therapy such as prevention of chronic pruritus (JP 2004-352714 A) and there is a prior art publication concerning the therapy of itching via the action of inhibiting the peripheral sensory nerves in the skin by selective inhibition of glutamic acid receptors (JP 2004-107209 A).

The itching of the skin is defined as “a sense of inclining to scratch the skin.” The dry state of the skin and the degree of itching generally show a positive correlation, and it is well known that the improvement of the dry state of the skin results in the reduction of itching. On the other hand, there is itching of the skin not accompanied by any abnormal skin property (“Knowledge of Diseases Common to Dermatology Necessary for Surgeons 6. Skin Pruritus and Mechanism of Itching (in Japanese),” (Shigeo Kochi), Rinsho Geka (=Clinical Surgery), Igaku-Shoin Ltd., Nov. 20, 2001, Vol. 56, No. 12, pages 1522-1524), and the relation between central itching and skin properties is not clear. The central itching caused by excessive reaction with the itching signals generated by the unbalance of intracerebral opioid peptides has nothing to do with abnormal properties of the skin.

Therefore, the prior art publications mentioned above do not disclose the skin property-improving effect of the active ingredient having a specific morphinan skeleton.

It could therefore be helpful to provide a novel medicine effective for improving skin properties such as preventing skin drying, improving skin roughening and improving the skin darkening involved in hyperkeratosis (thickened keratin layer) against the decline of the skin functions brought about by various causes.

### SUMMARY

We found that a compound having a specific morphinan skeleton or any of its pharmacologically permissible acid addition salts is useful as a skin property-improving therapeutic agent.

We thus provide the following [1] through [3]. We also provide a method of improving skin property, comprising administering an effective amount of a compound as the following [1] through [3]:

[1] Skin property-improving therapeutic agent comprising a compound represented by the following general formula (I):

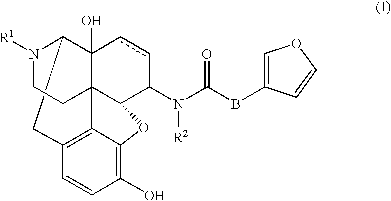

where the double line consisting of a dotted line and a solid line denotes a double bond or single bond; R¹ denotes a cycloalkylalkyl with 4 to 7 carbon atoms; R² denotes a straight chain or branched alkyl with 1 to 5 carbon atoms; and B denotes —CH═CH— or any of its pharmacologically permissible acid addition salts as an active ingredient.

[2] Skin property-improving therapeutic agent, according to [1], wherein in the general formula (I), R¹ denotes cyclopropylmethyl, cyclobutylmethyl, cyclopentylmethyl or cyclohexylmethyl; and R² denotes methyl, ethyl or propyl.

[3] Skin property-improving therapeutic agent, according to [1], wherein the compound represented by the general formula (I) is (+17-(cyclopropylmethyl)-3,14β-dihydroxy-4,5α-epoxy-6β[N-methyl-trans-3-(3-furyl)acrylamide]morphinan.

We thus provide a skin property-improving therapeutic agent comprising a morphinan derivative or any of its pharmacologically permissible acid addition salts as an active ingredient. Administration of such a skin property-improving therapeutic agent can improve skin properties, for example, can prevent skin drying, improve skin roughening and improve the skin darkening involved in hyperkeratosis.

### DETAILED DESCRIPTION

The skin property-improving therapeutic agent comprises as an effective component a compound represented by the Formula (1) and a pharmaceutically acceptable acid addition salt thereof:

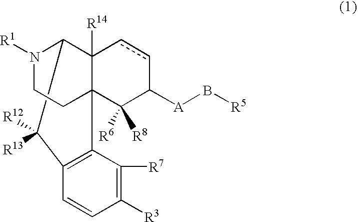

wherein the double line composed of a dashed line and a solid line represents a double bond or single bond with the latter being preferred;

R¹ represents C₁-C₅ alkyl, C₄-C₇ cycloalkylalkyl, C₅-C₇ cycloalkenylalkyl, C₆-C₁₂ aryl, C₇-C₁₃ aralkyl, C₄-C₇ alkenyl, allyl, furan-2-yl alkyl (wherein the number of carbon atoms in the alkyl moiety is 1 to 5) or thiophen-2-yl alkyl (wherein the number of carbon atoms in the alkyl moiety is 1 to 5);

R¹⁴ represents hydrogen, hydroxy, nitro, C₁-C₅ alkanoyloxy, C₁-C₅ alkoxy, C₁-C₅ alkyl, or NR⁹R¹⁰, wherein R⁹ represents hydrogen or C₁-C₅ alkyl; R¹⁰ represents hydrogen, C₁-C₅ or —C═O)R¹¹, and R¹¹ represents hydrogen, phenyl, or C₁-C₅ alkyl;

R³ represents hydrogen, hydroxy, C₁-C₅ alkanoyloxy or C₁-C₅ alkoxy;

A represents —XC(═Y)—, —XC(═Y)Z-, —X— or —XSO₂— (wherein X, Y and Z independently represent NR⁴, S or O, wherein R⁴ represents hydrogen, C₁-C₅ straight or branched alkyl or C₆-C₁₂ aryl, and in cases where more than one R⁴ are present in the formula, R⁴s may be the same or different);

B represents valence bond, C₁-C₁₄ straight or branched alkylene (wherein the alkylene may have at least one substituent selected from the group consisting of C₁-C₅ alkoxy, C₁-C₅ alkanoyloxy, hydroxy, fluorine, chlorine, bromine, iodine, amino, nitro, cyano, trifluoromethyl and phenoxy, and wherein 1 to 3 methylene groups therein may be replaced with carbonyl group(s)), C₂-C₁₄ straight or branched acyclic unsaturated hydrocarbon containing 1 to 3 double bonds and/or triple bonds (wherein the acyclic unsaturated hydrocarbon may have at least one substituent selected from the group consisting of C₁-C₅ alkoxy, C₁-C₅ alkanoyloxy, hydroxy, fluorine, chlorine, bromine, iodine, amino, nitro, cyano, trifluoromethyl and phenoxy, and that 1 to 3 methylene groups in the acyclic unsaturated hydrocarbon may be replaced with carbonyl group(s)), or C₁-C₁₄ straight or branched saturated or unsaturated hydrocarbon containing 1 to 5 thioether bonds, ether bonds and/or amino bonds (wherein a hetero atom does not directly binds to A, and 1 to 3 methylene groups are optionally replaced with carbonyl group(s));

R⁵ represents hydrogen or an organic group having a skeleton selected from those shown below:

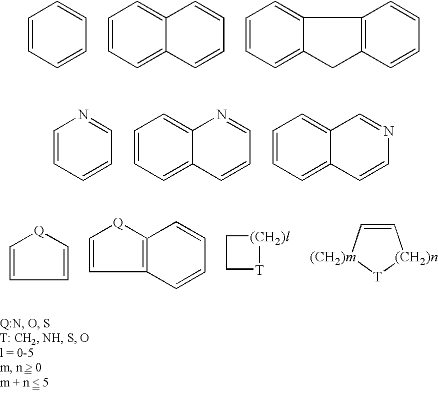

Organic groups represented by R⁵ (wherein Q represents N, O or S; T represents CH₂, NH, S or O; 1 represents an integer of 0 to 5; and m and n independently represent integers of 0 to 5, the total of m and n being not more than 5; each of the organic groups may have at least one substituent selected from the group consisting of C₁-C₅ alkyl, C₁-C₅ alkoxy, C₁-C₅ alkanoyloxy, hydroxy, fluorine, chlorine, bromine, iodine, amino, nitro, cyano, isothiocyanato, trifluoromethyl, trifluoromethoxy and methylenedioxy);

R⁶ represents hydrogen; R⁷ represents hydrogen, hydroxy, C₁-C₅ alkoxy or C₁-C₅ alkanoyloxy; or R⁶ and R⁷ together represent —O—, —CH₂— or —S—;

R⁸ represents hydrogen, C₁-C₅ alkyl or C₁-C₅ alkanoyl; and

R¹² and R¹³ both represent hydrogen, or one of them represents hydrogen and the other represents hydroxy, or they together represent oxo.

The Formula (I) includes (+), (−) and (±) isomers.

Among the compounds represented by the Formula (I), the skin property-improving compound preferably comprises as an effective component the compound represented by the already shown Formula (I) or the pharmaceutically acceptable acid addition salt thereof. The dashed line in the Formula (I) represents a double bond or single bond with the latter being preferred.

In the formula (I), R¹ denotes a cycloalkylalkyl with 4 to 7 carbon atoms. Above all, it is preferred that R¹ denotes cyclopropylmethyl, cyclobutylmethyl, cyclopentylmethyl or cyclohexylmethyl. It is especially preferred that R¹ denotes cyclopropylmethyl.

R² denotes a straight chain or branched alkyl with 1 to 5 carbon atoms. It is preferred that R² denotes methyl, ethyl or propyl. It is especially preferred that R² denotes methyl.

B denotes —CH═CH—. It is preferred that B denotes trans form —CH═CH—.

It is especially preferred that the compound represented by the formula (I) is a compound in which R¹ denotes cyclopropylmethyl; R² denotes methyl; and B denotes trans form —CH═CH—, namely, (−)-17-(cyclopropylmethyl)-3,14β-dihydroxy-4,5α-epoxy-6β[N-methyl-trans-3-(3-furyl)acrylamide]morphinan, though the compound of the general formula (I) is not limited to this specific compound.

These compounds represented by the Formula (I) may be produced by the method described in Japanese Patent No. 2525552. Among the compounds represented by the Formula (I), the compounds wherein both R¹² and R¹³ are hydrogen may be produced by the method described in Japanese Patent No. 2525552. Among the compounds represented by the Formula (1), the compounds wherein R¹² and R¹³ cooperatively represent oxo may be, for instance, produced by the method described in Chem. Pharm. Bull., 52, 664 (2004) and Japanese Patent No. 2525552 using a compound having 10-oxo obtained in accordance with literatures (Hererocycle, 63, 865 (2004); Bioorg. Med. Chem. Lett., 5, 1505 (1995)) as a raw material. In addition, among the compounds represented by the Formula (I), the compounds wherein R¹² is hydroxyl and R¹³ is hydrogen may be produced by the method described in Chem. Pharm. Bull., 52, 664 (2004).

Further, it is preferred that the skin property-improving therapeutic agent is used as a moisture retaining agent, skin barrier function restoration promoter, skin drying preventive and skin roughening preventive. It is desirable that the skin property-improving therapeutic agent is used as an oral medicine, but is not limited to such formulations, being able to be used as skin medicines for external application and the like unless the effects are impaired.

The pharmacologically permissible acid addition salts include inorganic acid salts such as hydrochlorides, sulfates, nitrates, hydrobromides, hydroiodides and phosphates, organic carboxylates such as acetates, lactates, citrates, oxalates, glutarates, malates, tartrates, fumarates, mandelates, maleates, benzoates and phthalates, organic sulfonates such as methanesulfonates, ethanesulfonates, benzenesulfonates, p-toluenesulfonates and camphorsulfonates and the like. Above all, hydrochlorides, hydrobromides, phosphates, tartrates, methanesulfonates and the like can be preferably used, though the acid addition salts are not of course limited to them.

Further, the compound represented by the formula (I) or any of its pharmacologically permissible acid addition salts is purified for medicinal application and subjected to a necessary safety test, and the compound that has passed the test can be orally administered as it is or as a medicinal composition obtained by mixing the compound with a publicly known pharmacologically permissible acid, carrier, excipient or the like. The formulation for oral administration can be selected from tablets, capsules, powder, granules and the like, although it is not of course limited to them. These formulations can be prepared by well-known methods usually used in the area of medicines. The dosage of the skin property-improving therapeutic agent can be adequately set based on the symptom, the age and weight of the patient, administration method and the like. Usually the amount of the active ingredient per adult per day is about 0.1 μg to about 100 mg in case of oral administration and about 0.01 μg to about 10 mg in case of non-oral administration.

Any of the skin property-improving therapeutic agents can be administered alone and can also be administered together with another drug, for example, a moisture retaining agent, anti-itching agent for external application, steroid ointment or the like. Examples of the moisture retaining agent include vaseline, urea, heparinoid ointment, Artemisia princes-mixed ointment, ceramide-containing cream and Camellia japonica-oil lotion. Examples of the anti-itching agent for external application include antihistamine ointment and crotamiton ointment. In the case where itching is strong, a steroid ointment can also be further used together. The skin property-improving therapeutic agents can be used as moisture retaining agents, skin barrier function restoration promoters, skin drying preventives and skin roughening preventives.

### Example

Our therapeutic agents are explained below in more detail with reference to an example.

### Example 1

2.5 μg of (−)-17-(cyclopropylmethyl)-3,14β-dihydroxy-4,5α-epoxy-6β[N-methyl-trans-3-(3-furyl)acrylamide]morphinan hydrochloride (compound 1) represented by the following structural formula (II) was hermetically contained in soft capsules formed of a gelatin film, to prepare an oral medicine. Two capsules (5.0 μg) of the oral medicine were administered to each of two itch patients treated with hemodialysis and having resistance against the conventional treatment. After start of administration, one patient (female, age 79) began to feel less itching and became smoother on the skin in general (especially legs), to have beautiful skin. The other patient (male, age 69) who had been very poor in complexion and skin texture became less dry on the face and gained good complexion after start of administration with the oral medicine. About 4 to 5 days after start of administration with the oral medicine, when one of the patients was relieved of the itching felt on the portions ranging from the wrists to the shoulders and on the breasts, the patient began to have moist skin, but after end of administration, when the patient began to feel itching again, the patient began to have dry skin.

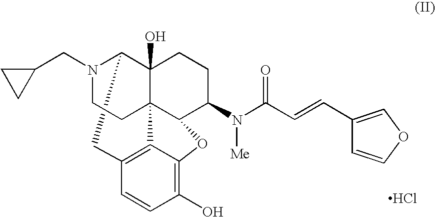

### INDUSTRIAL APPLICABILITY

Our therapeutic agents are useful as a skin property-improving therapeutic agent that can prevent skin roughening, skin drying, skin darkening and the like and give a moisture retaining effect.

1. A method of improving skin property in a subject, comprising administering an effective amount of a compound represented by the following formula (I): 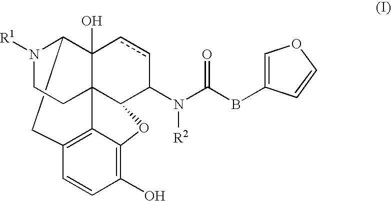
where the double line consisting of a dotted line and a solid line denotes a double bond or single bond; R¹ denotes a cycloalkylalkyl with 4 to 7 carbon atoms; R² denotes a straight chain or branched alkyl with 1 to 5 carbon atoms; and B denotes —CH═CH— or any of its pharmacologically permissible acid addition salts the subject.

2. The method of claim 1, wherein in the formula (I), R¹ denotes cyclopropylmethyl, cyclobutylmethyl, cyclopentylmethyl or cyclohexylmethyl; and R² denotes methyl, ethyl or propyl.

3. The method of claim 1, wherein the compound represented by the formula (I) is (−)-17-(cyclopropylmethyl)-3,14β-dihydroxy-4,5α-epoxy-6β[N-methyl-trans-3-(3-furyl)acrylamide]morphinan.

4. (canceled)

5. The method of claim 1, wherein the acid addition salts are selected from the group consisting of hydrochlorides, sulfates, nitrates, hydrobromides, hydroiodides and phosphates, organic carboxylates such as acetates, lactates, citrates, oxalates, glutarates, malates, tartrates, fumarates, mandelates, maleates, benzoates, phthalates and organic sulfonates.

6. The method of claim 1, wherein the compound (I) is combined with at least one of a carrier or excipient.

7. The method of claim 1, wherein the compound (I) is in the form of tablets, capsules, powder or granules.

8. The method of claim 1, wherein the compound (I) is orally administered in an amount of about 0.1 μg to about 10 mg/day.

9. The method of claim 1, wherein the compound (I) is non-orally administered in an amount of about 0.01 μg to 10 mg/day.

10. The method of claim 1, further comprising administering another therapeutic agent.

11. The method of claim 10, wherein the therapeutic agent is a moisture retaining agent, an anti-itching agent or a steroid.
